(19) United States
(12) Patent Application Publication
(10) Pub. No.: US 2012/0264193 A1
(43) Pub. Date: Oct. 18, 2012

(54) METHOD FOR PRODUCING MEDIUM AND MEDIUM PRODUCED THEREBY

(76) Inventors: Rumiko Kuwana, Kanagawa (JP); Atsuhiro Sagitani, Kanagawa (JP); Taketo Wakai, Kanagawa (JP); Hiroaki Goto, Kanagawa (JP)

(21) Appl. No.: 13/439,899

(22) Filed: Apr. 5, 2012

Related U.S. Application Data
(60) Provisional application No. 61/475,339, filed on Apr. 14, 2011.

(51) Int. Cl. C12N 1/20

(52) U.S. Cl. 435/252.1

(57) ABSTRACT

[Problem to be Solved]

Provided is a method for producing a medium which decreases loss of nutritional components due to an interaction between the medium nutritional components, which interaction is mediated by a Maillard reaction, etc.

[Solution]

A method for producing a medium for culturing microbes is used, the method including the steps of:

(1) sterilizing a solution comprising a sugar source material;
(2) sterilizing a solution comprising a nitrogen source material; and
(3) blending the two solutions as obtained in steps (1) and (2).

### TECHNICAL FIELD

The present invention relates to media for culturing microbes and media for producing an immunomodulator.

### BACKGROUND ART

Medium sterilization is carried out so as to prevent unwanted-microbe contamination. However, the medium sterilization has an adverse reaction due to heat during sterilization, which causes loss of medium nutritional components included. As a reaction inducing the loss of nutritional components, widely known are an interaction between medium nutritional components and destruction of components having low heat-resistance. In particular, as the interaction between medium nutritional components, a browning phenomenon, what is called a Maillard reaction, is known that causes not only coloring of a medium, but also destruction of medium components. Such a reaction results from a combination of a carbonyl group included in a medium and an amino group of amino acids and proteins (Non Patent Literature 1, see p. 96).

In addition, WO2006/073145 (Patent Literature 1), for example, discloses a composition of a conventional medium.

In the meantime, a correlation between unwanted-microbe killing and an activation energy for nutritional component destruction has revealed as follows: compared to batch sterilization such as autoclave sterilization and batch disinfection, continuous sterilization, which allows for ultra-high-temperature and short-time (UHT) sterilization, can inhibit the nutritional component destruction while keeping a degree of sterilization sufficient to kill the unwanted microbes. The continuous sterilization is not susceptible to a scale-up effect, so that this becomes an additional advantage (Non Patent Literature 1, see p. 96 to 102).

Unfortunately, there are a few cases of industrial equipment having a continuous sterilizer. In most cases, batch disinfection is employed. In addition, no insight has been found on a method for sterilizing a medium for producing microbes having an immunoregulatory function such as an IL-12-inducing function. This function should be exerted at the time of ingesting the microbes by a human.

### CITATION LIST

### Patent Literature

WO2006/073145

### Non-Patent Literature

“Principles of Fermentation Technology, from Laboratory to Factory” by P. F. Stanbury and A. Whitaker, 1988, published by Japan Scientific Societies Press.

### SUMMARY OF INVENTION

A method for producing a medium for culturing microbes, comprising the steps of:
(1) sterilizing a solution comprising a sugar source material;
(2) sterilizing a solution comprising a nitrogen source material; and
(3) blending the two solutions as obtained in steps (1) and (2).

### Technical Problem

The present invention provides a method which decreases loss of nutritional components due to an interaction between the medium nutritional components, which interaction is mediated by a Maillard reaction, etc., and/or a method for carrying out batch sterilization of a medium by batch disinfection, etc., and a method for carrying out continuous sterilization of a medium by ultra-high-temperature and short-time sterilization, etc. In addition, the present invention provides a method for producing a medium for production of microbes having an immunoregulatory function such, as an IL-12-inducing function. This function should be exerted at the time of ingesting the microbes by a human.

### Solution to Problem

The present inventors have found a solution of the above problems by sterilizing, independently, a solution comprising a nitrogen source material and a solution comprising a sugar source material, and thereafter by blending the solutions.

The present inventors also have found a solution of the above problems by sterilizing, independently, a solution comprising a sugar and a solution comprising a nitrogen source, and thereafter by blending the solutions.

Specifically, the present invention provides a method for producing a medium for culturing microbes, comprising the steps of: (1) sterilizing a solution comprising a sugar source material; (2) sterilizing a solution comprising a nitrogen source material; and (3) blending the two solutions as obtained in steps (1) and (2).

The present invention also provides a method for producing a medium for culturing microbes, comprising the steps of: (1) sterilizing a solution devoid of a nitrogen source, the solution comprising a sugar; (2) sterilizing a solution devoid of a sugar, the solution comprising a nitrogen source; and (3) blending the two solutions as obtained in steps (1) and (2).

The present invention also provides a method for producing a medium for culturing microbes, comprising the steps of: (1) sterilizing a solution solely comprising a sugar; (2) sterilizing a solution solely comprising a nitrogen source; (3) sterilizing a solution devoid of a sugar or a nitrogen source, the solution comprising at least one selected from the group consisting of inorganic salts, vitamins, fatty acids, buffers, and antifoaming agents; and (4) blending the three solutions as obtained in steps (1), (2), and (3).

The present invention also provides the method for production, wherein the sugar source material or sugar is a nonreducing sugar.

The present invention also provides the method for production, wherein the nonreducing sugar comprises at least one selected from the group consisting of sucrose, trehalose, kestose, melezitose, gentianose, neobifurcose, fungitetraose, and bifurcose.

The present invention also provides the method for production, wherein the nonreducing sugar is sucrose.

The present invention also provides the method for production, wherein the nitrogen source material or nitrogen source comprises at least one selected from the group consisting of amino acids, peptides, proteins, urea, casein hydrolysates, corn steep liquor, soy bean, soy bean hydrolysates, peanut meal, cotton seed meal, fish meal, yeast extract, and fish extract.

The present invention also provides the method for production, wherein the step of sterilizing a solution comprising a sugar source or the step of sterilizing a solution comprising a sugar is carried out by batch sterilization and/or continuous sterilization.

The present invention also provides the method for production, wherein the step of sterilizing a solution comprising a nitrogen source material or the step of sterilizing a solution comprising a nitrogen source is carried out by batch sterilization and/or continuous sterilization.

The present invention also provides the method for production, wherein batch sterilization and/or continuous sterilization is carried out in the step of sterilizing a solution devoid of a sugar or a nitrogen source, the solution comprising at least one selected from the group consisting of inorganic salts, vitamins, fatty acids, buffers, and antifoaming agents.

The present invention also provides a medium which is produced by the above method for production.

The present invention also provides a method for culturing microbes, comprising the step of using a medium produced by the above method for production.

The present invention also provides the method for culture, wherein the microbes are lactic acid bacteria.

The present invention also provides microbes which are cultured by the above method for culture.

The present invention also provides lactic acid bacteria which are cultured by the above method for culture.

The present invention also provides a method for producing a medium for producing an immunomodulator, comprising the steps of: (1) sterilizing a solution comprising a sugar source material; (2) sterilizing a solution comprising a nitrogen source material; and (3) blending the two solutions as obtained in steps (1) and (2).

The present invention also provides a method for producing a medium for producing an immunomodulator, comprising the steps of: (1) sterilizing a solution devoid of a nitrogen source, the solution comprising a sugar; (2) sterilizing a solution devoid of a sugar, the solution comprising a nitrogen source; and (3) blending the two solutions as obtained in steps (1) and (2).

The present invention also provides a method for producing a medium for producing an immunomodulator, comprising the steps of: (1) sterilizing a solution solely comprising a sugar; (2) sterilizing a solution solely comprising a nitrogen source; (3) sterilizing a solution devoid of a sugar or a nitrogen source, the solution comprising at least one selected from the group consisting of inorganic salts, vitamins, fatty acids, buffers, and antifoaming agents; and (4) blending the three solutions as obtained in steps (1), (2), and (3).

The present invention also provides the method for production, wherein the sugar source material or sugar is a nonreducing sugar.

The present invention also provides the method for production, wherein the nonreducing sugar comprises at least one selected from the group consisting of sucrose, trehalose, kestose, melezitose, gentianose, neobifurcose, fungitetraose, and bifurcose.

The present invention also provides the method for production, wherein the nonreducing sugar is sucrose.

The present invention also provides the method for production, wherein the nitrogen source material or nitrogen source comprises at least one selected from the group consisting of amino acids, peptides, proteins, urea, casein hydrolysates, corn steep liquor, soy bean, soy bean hydrolysates, peanut meal, cotton seed meal, fish meal, yeast extract, and fish extract.

The present invention also provides the method for production, wherein the step of sterilizing a solution comprising a sugar source or the step of sterilizing a solution comprising a sugar is carried out by batch sterilization and/or continuous sterilization.

The present invention also provides the method for production, wherein the step of sterilizing a solution comprising a nitrogen source material or the step of sterilizing a solution comprising a nitrogen source is carried out by batch sterilization and/or continuous sterilization.

The present invention also provides the method for production, wherein batch sterilization and/or continuous sterilization is carried out in the step of sterilizing a solution devoid of a sugar or a nitrogen source, the solution comprising at least one selected from the group consisting of inorganic salts, vitamins, fatty acids, buffers, and antifoaming agents.

The present invention also provides a medium which is produced by the above method for production.

The present invention also provides a method for producing an immunomodulator, comprising the step of using the above medium.

The present invention also provides the method for production, wherein the immunomodulator is an antiallergic agent.

The present invention also provides the method for production, wherein the immunomodulator is an activator for inducing IL-12.

The present invention also provides an immunomodulator which is produced by the above method for production.

The present invention also provides an antiallergic agent which is produced by the above method for production.

The present invention also provides an activator for inducing IL-12, the activator being produced by the above method for production.

### ADVANTAGEOUS EFFECTS OF INVENTION

The present invention provides a method which decreases loss of nutritional components due to an interaction between the medium nutritional components, which interaction is mediated by a Maillard reaction, etc., and/or a method for carrying out batch sterilization of a medium by batch disinfection, etc., and a method for carrying out continuous sterilization of a medium by ultra-high-temperature and short-time sterilization, etc. A medium according to the present invention has a good color tone of the medium by itself, and also has an excellent characteristic of culturing microbes. In addition, use of a medium according to the present invention enables an immunomodulator such as an activator for inducing IL-12 to be efficiently produced. Furthermore, use of a medium according to the present invention allows for production of microbes or an immunomodulator having a good color tone.

### BRIEF DESCRIPTION OF DRAWINGS

FIG. 1 is a graph showing changes in turbidity during culture.

FIG. 2 is a graph showing activities of inducing IL-12.

FIG. 3 is a graph showing changes in turbidity during culture.

FIG. 4 is a graph showing activities of inducing IL-12.

FIG. 5 is a graph showing changes in turbidity during culture.

FIG. 6 is a graph showing activities of inducing IL-12 by microbial cells which have been cultured at a scale of 4.2 t.

FIG. 7 is a graph showing changes in turbidity during culture.

### DESCRIPTION OF EMBODIMENTS

A sugar source material or sugar which can be used in the present invention is not particularly limited. Any of reducing sugars and nonreducing sugars having no reducibility can be used, but the nonreducing sugars are preferable.

Examples of the reducing sugars can include glucose, pyranose, aldohexose, furanose, ketopyranose, ketohexose, ketofuranose, and the like.

Examples of the nonreducing sugars can include sucrose, trehalose, kestose, melezitose, gentianose, neobifurcose, fungitetraose, bifurcose, and the like. Among the nonreducing sugars, sucrose is preferable.

A nitrogen source material or nitrogen source which can be used in the present invention is not particularly limited if they can supply a medium with nitrogen. Examples of them can include amino acids, peptides, proteins, urea, and the like. Examples of the natural nitrogen source which can be used as a raw material for a medium can include casein hydrolysates, corn steep liquor, soy bean and soy bean hydrolysates, peanut meal, cotton seed meal, fish meal, fish extract, beef extract, yeast extract, and the like.

Examples of the casein hydrolysates can include, but are not limited to, milk casein that has been digested by pepsin or pancreatin. Specific examples can include “the product name: Casein Peptone Plus” which is commercially available from Organotechnie, Inc., and the like.

The fish extract is not particularly limited if it is prepared from fish meat. Examples of the fish extract can include “the product name: Bacterio-N-KS(B)” which is commercially available from Maruha Nichiro Seafoods, Inc., and the like.

Examples of the beef extract can include, but are not limited to, “the product name: Meast Peptone” which is commercially available from Primatone RL, Inc., and the like.

The yeast extract is not particularly limited if it has been extracted from yeast media. Examples of the yeast extract can include “the product name: YP21 CM” which is commercially available from Fuji Foods Corporation, “the product name: SK yeast extract HUP-2” which is commercially available from NIPPON PAPER CHEMICALS, “the product name: Yeast Peptone Standard Type F” which is commercially available from Organotechnie, Inc., and the like.

As used herein, additional components of a medium except for a sugar source material, sugar, a nitrogen source material, and a nitrogen source are not particularly limited if the components can be usually used in a medium. Examples of the additional components can include inorganic salts, vitamins, fatty acids, buffers, antifoaming agents, and the like.

Examples of the inorganic salts can include magnesium sulfate, dipotassium hydrogenphosphate, calcium carbonate, manganese sulfate, copper sulfate, zinc sulfate, iron sulfate, and the like.

Examples of the vitamins can include ascorbic acid, thiamine, biotin, sodium pantothenate, folic acid, nicotinic acid amide, riboflavin, niacin, pyridoxine, inositol, and the like.

Examples of the fatty acids can include higher fatty acid monoglyceride which is included in a palm or rapeseed oil, medium chain fatty acid monoglyceride, polyglycerin fatty acid ester, and the like. Examples of the polyglycerin fatty acid ester can include decaglycerin monooleate, diglycerin monodioleate, decaglycerin decaoleate, and the like.

Examples of the buffers can include organic acids such as sodium acetate, inorganic acids such as dipotassium hydrogenphosphate and calcium carbonate, marble, and the like.

Examples of the antifoaming agents can include polyglycerin fatty acid esters such as decaglycerin monooleate.

As used herein, examples of a solvent which can dissolve the respective components of a medium can include water. Specific examples of the water which can be used include purified water, deionized water, distilled water, sterilized water, tap water, and the like.

As used herein, the “solution comprising a sugar source material” can comprise, in addition to the above sugars, any of other medium components such as inorganic salts, vitamins, fatty acids, buffers, and antifoaming agents. Furthermore, the above solution means a solution optionally comprising a nitrogen source whose amount is allowed as long as effects of an invention of the present application can be exerted by this amount.

Here, the amount of the nitrogen source which can be allowed as long as effects of an invention of the present application can be exerted by this amount is not particularly limited if the amount is an amount to achieve the effects of an invention of the present application. However, the amount may be usually 10% by weight or less per total amount of a “solution comprising a sugar source material”, preferably 5% by weight or less, more preferably 1% by weight or less, still more preferably 0.1% by weight or less, still more preferably 0.01% by weight or less, and most preferably 0%.

As used herein, the “solution comprising a nitrogen source material” can comprise, in addition to the above nitrogen source, any of other medium components such as inorganic salts, vitamins, fatty acids, buffers, and antifoaming agents. Furthermore, the above solution means a solution optionally comprising a sugar whose amount is allowed as long as effects of an invention of the present application can be exerted by this amount.

Here, the amount of the sugar which can be allowed as long as effects of an invention of the present application can be exerted by this amount is not particularly limited if the amount is an amount to achieve the effects of an invention of the present application. However, the amount may be usually 10% by weight or less per total amount of a “solution comprising a nitrogen source material”, preferably 5% by weight or less, more preferably 1% by weight or less, still more preferably 0.1% by weight or less, still more preferably 0.01% by weight or less, and most preferably 0%.

As used herein, the “solution comprising a sugar devoid of a nitrogen source” means both the case of a solution comprising a sugar and another medium component (excluding a nitrogen source) and the case of a solution solely comprising a sugar.

In a similar manner, as used herein, the “solution comprising a nitrogen source devoid of a sugar” means both the case of a solution comprising a nitrogen source and another medium component (excluding a sugar) and the case of a solution solely comprising a nitrogen source.

As used herein, the step of sterilizing a “solution comprising a sugar source material, a “solution comprising a nitrogen source material”, or a solution comprising a nitrogen source, a sugar, and another component is not particularly limited if the step is a step of inactivating (sterilizing) microbes present in a solution. Examples of a heat sterilization step can usually include a batch sterilization step and a continuous sterilization step.

In the batch sterilization, any of sterilization using an autoclave and batch disinfection using a steam injection process, etc., can be carried out. In the continuous sterilization, ultra-high-temperature and short-time (UHT) sterilization of a plate type or a tube type, etc., can be carried out.

The temperature in the case of the batch sterilization is appropriately determined depending on medium components included in a sterilization subject, but is usually in a range of 80 to 150° C. and preferably 100 to 130° C.

The duration in the case of the batch sterilization is appropriately determined depending on medium components included in a sterilization subject, but is usually in a range of 5 to 180 minutes and preferably 15 to 100 minutes.

The temperature in the case of the continuous sterilization is appropriately determined depending on medium components included in a sterilization subject, but is usually in a range of 80 to 200° C. and preferably 100 to 160° C.

The duration in the case of the continuous sterilization is appropriately determined depending on medium components included in a sterilization subject, but is usually in a range of 5 to 180 seconds and preferably 10 to 100 seconds.

Furthermore, in the case of carrying out either the batch sterilization or the continuous sterilization, sterilization can be allowed not only at a laboratory scale of several mL to several L, but also at a pilot plant scale or commercial plant scale of 1 to 100 t as a medium volume for a sterilization subject.

Any of the above autoclave sterilization and ultra-high-temperature and short-time (UHT) sterilization has no particular limitation concerning a degree of sterilization. However, the degree may be usually in a range of F0=1 to 50 and preferably about F0=10 to 30. Also, examples of a method for regulating a degree of sterilization can include a method using a thermo processor.

As used herein, the respective two and three solutions which have been sterilized can be blended to produce a medium of the present invention. The blending method is not particularly limited, but, for example, the respective sterilized solutions are poured into a culture vessel and blended by stirring with a mixer.

In addition, in the case of blending each sterilized solution, an alkali agent such as sodium hydroxide is slowly added to adjust the pH to 4.0 to 8.0 and preferably about 6.0 to 7.5, and a medium of the present invention can be then prepared. When the above pH remains within a predetermined pH, the pH adjustment is not necessary.

A microbe which can be cultured in a medium of the present invention is not particularly limited. However, examples of the microbe can include lactic acid bacteria, bacteria which belong to Bifidobacterium, yeasts, molds (Aspergillus), and the like.

An immunomodulator which can be produced using a medium of the present invention is not particularly limited if the immunomodulator has an immune regulatory effect. Examples of the immunomodulator can include an antiallergic agent, an activator for inducing IL-12, and the like. Examples of a method for producing an immunomodulator according to the present invention can include a method comprising the step of: culturing microbes by using a medium of the present invention; and isolating an immunomodulator after the culture. In addition, depending on usage forms, an immunomodulator may not be isolated, and a culture mixture may be filtered and/or dried, or a cultured medium may be used as it is.

An immunomodulator according to the present invention can be used as an antiallergic agent, an IgE-production inhibitor, an atopy reduction/treatment/prophylaxis agent, a pollinosis reduction/treatment/prophylaxis agent, a perennial allergy reduction/treatment/prophylaxis agent, an asthma reduction/treatment/prophylaxis agent, house dust allergen reduction/treatment/prophylaxis agent, and the like.

Microbes which have been produced by using the present invention can be provided as an immunomodulator, including viable cells, dried viable cells, sterilized cells, cell homogenates, and the like. The microbe can be provided as a beverage, diet, or supplement containing such an immunomodulator, etc.

Here, examples of a microbe which can be used to produce an immunomodulator can include, but are not limited to, lactic acid bacteria, Lactobacillus bifidus, yeasts, molds (Aspergillus), and the like. Among them, the lactic acid bacteria are preferable. In particular, lactic acid bacteria which belong to genus Lactobacillus are preferable. Preferred is Lactobacillus acidophilus, and particularly preferred is a Lactobacillus acidophilus L-92 strain (deposited at a Japan incorporated administrative agency, National Institute of Advanced Industrial Science and Technology, Patent Microorganisms Depositary, as a Lactobacillus acidophilus CL-92 strain, the deposit number: FERM BP-4981). In addition, exemplified examples can include a Lactobacillus acidophilus CL-0062 strain (deposited at a Japan incorporated administrative agency, National Institute of Advanced Industrial Science and Technology, Patent Microorganisms Depositary as the deposit number: FERM BP-4980), a Lactobacillus fermentum CP-34 strain (deposited at a Japan incorporated administrative agency, National Institute of Advanced Industrial Science and Technology, Patent Microorganisms Depositary as the deposit number: FERM BP-8383), and the like.

Examples of the lactic acid bacteria which can be used in a culture method according to the present invention can include Lactobacillus delbrueckii subsp. bulgaricus. The specific examples can be listed below.

Examples of the lactic acid bacteria can include genus Lactobacillus, genus Bifidobacterium, genus Enterococcus, genus Leuconostoc, genus Streptococcus, genus Lactococcus, genus Pediococcus, genus Weissella, and the like.

Examples of the lactic acid bacteria which belong to the above genus Lactobacillus can include Lactobacillus amylovorus, Lactobacillus gasseri, Lactobacillus casei, Lactobacillus paracasei, Lactobacillus zeae, Lactobacillus rhamnosus, Lactobacillus reuteri, Lactobacillus acidophilus, Lactobacillus crispatus, Lactobacillus gallinarum, Lactobacillus brevis, Lactobacillus fermentum, Lactobacillus plantarum, Lactobacillus delburueckii subsp. bulgaricus, Lactobacillus johnsonii, and the like.

Examples of the bacteria which belong to the above genus Bifidobacterium can include Bifidobacterium breve, Bifidobacterium longum, Bifidobacterium pseudolongum, Bifidobacterium animalis, Bifidobacterium adolescentis, Bifidobacterium bifidum, Bifidobacterium lactis, Bifidobacterium catenulatum, Bifidobacterium pseudocatenulatum, Bifidobacterium magnum, and the like

Examples of the bacteria which belong to the above genus Enterococcus can include Enterococcus faecalis, Enterococcus faecium, and the like.

Examples of the bacteria which belong to the above genus Streptococcus can include Streptococcus thermophilus, Streptococcus lactis, Streptococcus diacetilactis, Streptococcus faecalis, and the like

Examples of the bacteria which belong to the above genus Leuconostoc can include Leuconostoc mesenteroides, Leuconostoc lactis, and the like.

Examples of the bacteria which belong to the above genus Lactococcus can include Lactococcus lactis, Lactococcus plantarum, Lactococcus raffinolactis, and the like.

Examples of the bacteria which belong to the above genus Pediococcus can include Pediococcus pentosaceus, Pediococcus damnosus, and the like.

Examples of the bacteria which belong to the above genus Weissella can include Weissella cibaria, Weissella confusa, Weissella halotolerans, Weissella hellenica, Weissella kandleri, Weissella kimchii, Weissella koreensis, Weissella minor, Weissella paramesenteroides, Weissella soli, Weissella thailandensis, Weissella viridescens, and the like.

### EXAMPLES

Hereinafter, the present invention is more specifically described by using Examples. However, these Examples do not limit the present invention.

### Examples 1 to 3

### Sterilization of Sugar (Sucrose) Alone

### (1) Medium Preparation (Example 1, Comparative Example 1)

According to a formulation as designated in Table 1, the respective components were dissolved in purified water to prepare 500 mL of a medium. This medium was poured into a jar fermenter (Model: BMJ-01, manufactured by Able Corp.), and was sterilized using an autoclave (Model: LBS-325, manufactured by Tomy Corp.) at 121° C. for 90 minutes. After that, the pH was adjusted to 6.8 by using about 50% by weight of aqueous sodium hydroxide (which complies with food additive standards) (Comparative Example 1).

In addition, among the components listed in Table 1, 35 g of sucrose was solely dissolved in 165 mL of purified water to yield a sucrose solution. Also, components listed in Table 1 except for sucrose were dissolved in purified water to 300 mL to yield a mixed solution containing medium components other than sucrose. Next, the above sucrose solution and the mixed solution containing medium components other than sucrose were each independently sterilized using an autoclave at 121° C. for 90 minutes. Then, these solutions were blended and the mixture was filled to the mark with sterilized water (in which purified water was sterilized using an autoclave at 121° C. for 20 minutes) to yield 500 mL of a medium. After that, the pH was adjusted to 6.8 by using about 50% by weight of aqueous sodium hydroxide (which complies with food additive standards) (Example 1).

TABLE 1
Formulation Amount of Medium Components

| | Formulation |
|---|---|
| Medium components | ratio (w/v %) |
| Sucrose | 7.000 |
| Fish extract (Product Name: Bacterio-N-KS(B), | 3.000 |
| manufactured by Maruha Nichiro Seafoods, Inc.) | |
| Casein peptone (Product Name: Casein Peptone Plus, | 1.500 |
| manufactured by Organotechnie, Inc.) | |
| Yeast extract (Product Name: Yeast Peptone Standard | 5.000 |
| Type F, manufactured by Organotechnie, Inc.) | |
| Decaglycerin monooleate (Product Name: Sunsoft Q-17S, | 0.300 |
| manufactured by Taiyo Kagaku Co., Ltd.) | |
| Sodium acetate trihydrate (which complies with food | 0.500 |
| additive standards) | |
| Dipotassium hydrogenphosphate (which complies with | 0.445 |
| food additive standards) | |
| Magnesium sulfate heptahydrate (which complies with food | 0.200 |
| additive standards) | |
| Purified water | 82.055 |
| Total | 100.000 |

With regard to the medium, by using a medium color tone as an index, light absorbance at 600 nm (OD600) of a medium supernatant was determined (by using a spectrophotometer (the device name: Nanophotometer, manufactured by Implen). Table 2 shows the results.

TABLE 2
Medium Sterile Conditions and Color Tone after Sterilization

| | Sterile | | Color tone |
|---|---|---|---|
| | conditions | Sterilization procedure | (OD600) |
| Comparative | 121° C., 90 min | Blend all the | 0.8 |
| Example 1 | | components, and sterilize | |
| Example 1 | 121° C., 90 min | Separately sterilize a | 0.6 |
| | | sugar component and the | |
| | | other components | |

Table 2 demonstrated that the medium of Example 1 had a lower absorbance than the medium of Comparative Example 1. This suggests that the browning is suppressed.

### (2) Culture of Lactic Acid Bacteria (Example 2, Comparative Example 2)

By using the above respective media of Comparative Example 1 and Example 1, lactic acid bacteria, a Lactobacillus acidophilus L-92 strain (FERM BP-4981), were cultured. The lactic acid bacteria which had been cultured using MRS medium (the product name: Lactobacilli MRS broth, manufactured by BD) were used. Next, 1 to 5% of the lactic acid bacteria were aseptically inoculated in 500 mL of the respective media as obtained in Comparative Example 1 and Example 1. Until the pH reached 4.5 or lower (i.e., it took about 30 hours), the mixtures were each cultured at a temperature of 37° C. with stirring. A cultured medium was sampled over time, and the turbidity (OD600) was determined using a spectrophotometer (the device name: Nanophotometer, manufactured by Implen) to check a rough indication for cell number. A cultured medium at the end of the culture was centrifuged using a centrifuge (the device name: Universal Cold Centrifuge 5910, manufactured by KUBOTA Corporation) at about 2500 G for 10 minutes to collect a precipitate (microbial cells). The resulting microbial cells were washed with 500 mL of purified water, and then dried using a lyophilizer (Model: FDU-830, manufactured by EYELA) to be further powderized. Regarding the resulting respective powder, a cell content (g/L) per medium and its color tone were determined.

The cell content per medium was calculated by measuring the weight of the lyophilized powder.

In addition, with regard to the color tone, the Lab color space was determined using a colorimeter (Model: CM-3500d, manufactured by Konica Minolta Holdings, Inc.), and the L value was designated as an index for color rendering. Table 3 shows the results.

TABLE 3
Cell yield and Color Tone of Powder of Microbial
Cells Cultured by Using

| Each Medium | | | |
|---|---|---|---|
| Cultured | | | |
| lactic acid | | Cell content | Color tone |
| bacteria | Medium | (g/L) | (L Value) |
| Comparative | Medium of Comparative | 3.7 | 61.2 |
| Example 2 | Example 1 | | |
| Example 2 | Medium of Example 1 | 5.0 | 64.1 |

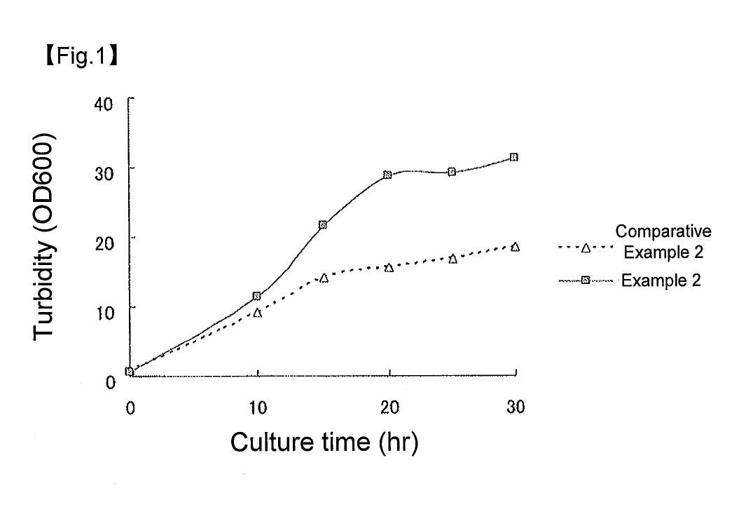

FIG. 1 and Table 3 demonstrated that lactic acid bacteria (Example 2) which had been cultured using the medium of Example 1 had a higher growth rate of the lactic acid bacteria and also had a higher cell yield than those (Comparative Example 2) which had been cultured using the medium of Comparative Example 1. In addition, regarding the color tone indicated by the L value, the lactic acid bacteria of Example 2 had a larger L value, which demonstrated that the lactic acid bacteria of Example 2 had less browning than the lactic acid bacteria of Comparative Example 2.

### (3) Determination of Activities of Inducing IL-12 (Example 3, Comparative Example 3)

By using the above respective lactic acid bacteria which had been cultured in Comparative Example 2 and Example 2, activities of inducing IL-12 were determined.

Preparation of Splenocytes:

0.1 mL of an OVA solution (in which 1 mg of OVA (produced by SIGMA), 1 mL of PBS(−), and 1 mL of Imject Alum (manufactured by Thermo) were suspended) per mouse was intraperitoneally administered to BALB/c mice (7- to 9-week-old males, supplied by Charles River Laboratories Japan Inc.). After rearing for 10 to 12 days, the mice were sacrificed by performing cervical dislocation, and their spleen was surgically removed. The spleen was suspended in RPMI modified medium (RPMI 1640 medium (manufactured by Invitrogen) containing 10% FBS and 100 U/mL penicillin/100 μg/mL streptomycin (manufactured by Invitrogen)), and was made to pass through a 70 μm cell strainer (manufactured by FALCON) to result in single cells. The single cells were suspended in a hemolysis solution and centrifuged. After the supernatant was removed, the single cells were diluted with RPMI modified medium to have the viable cell number of 5.0×10⁶ cells/mL. Finally, a splenocyte suspension was thus prepared.

Coculture of Splenocytes and Lactic Acid Bacteria Powder:

To a 96-well flat-bottom plate (manufactured by FALCON) were added per well the above splenocyte suspension, each lactic acid bacteria powder as obtained in Comparative Example 2 or Example 2, and OVA of 200 μL, 1 μg, and 20 n, respectively, and the mixture was cultured at 37° C. under a 5% CO₂ atmosphere for 24 hours.

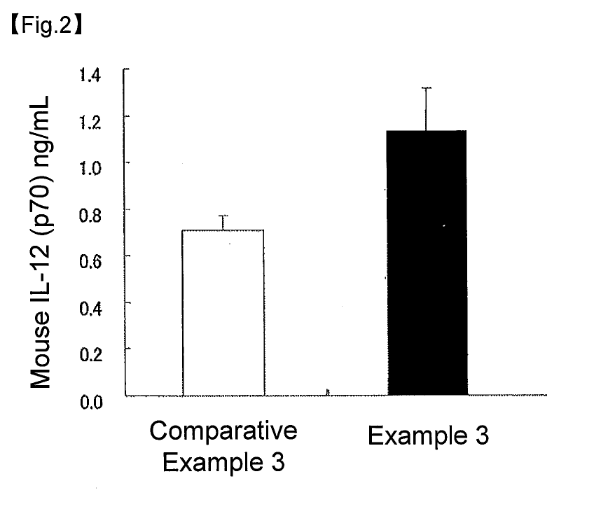

IL-12 determination: IL-12 whose production had been induced in the foregoing cultured medium was determined (FIG. 2, n=6) using a mouse IL-12 p70 measurement kit (the product name: OptElA Mouse IL-12(p70) Kit, manufactured by BD) and a 96-well immunoassay plate (manufactured by Nunc).

FIG. 2 demonstrated that the lactic acid bacteria (Example 2) which had been cultured using the medium (Example 1) prepared by separately sterilizing a sucrose component and the other components and thereafter by blending them had a remarkably higher activity of inducing IL-12 (Example 3) than an activity of inducing IL-12 (Comparative Example 3) of the lactic acid bacteria (Comparative Example 2) which had been cultured using the medium (Comparative Example 1) prepared by blending all the medium components and thereafter by sterilizing them.

### Examples 4 and 5

### Sterilization of Nitrogen Source (Yeast Extract) Alone

### (1) Medium Preparation, Part I (Example 4)

Among the medium components as designated in Table 4, 25 g of yeast extract (YP-21CM) and 10 g of yeast extract (HUP-2) were only dissolved in 115 mL of purified water to yield a yeast extract solution. In addition, medium components other than yeast extract were dissolved in purified water to 300 mL to yield a mixed solution containing the medium components other than yeast extract. Then, the above yeast extract solution was sterilized using an autoclave at 121° C. for 20 minutes. Also, after the mixed solution containing the medium components other than yeast extract had been sterilized using an autoclave at 121° C. for 120 minutes, these solutions were mixed and filled to the mark with sterilized water to yield 500 mL of a medium.

### (2) Medium Preparation, Part II (Example 5)

Among the medium components as designated in Table 4, 25 g of yeast extract (YP-21CM) and 10 g of yeast extract (HUP-2) were only dissolved in 115 mL of purified water to yield a yeast extract solution. In addition, among the medium components listed in Table 4, 45 g of sucrose was solely dissolved in 55 mL of purified water to yield a sucrose solution. Further, purified water was added to medium components other than yeast extract and sucrose to have 200 mL of a solution, and a mixed solution containing the medium components other than yeast extract and sucrose was obtained. Then, the above yeast extract solution was sterilized using an autoclave at 121° C. for 20 minutes. In addition, the sucrose solution was sterilized using an autoclave at 121° C. for 120 minutes. Furthermore, the above mixed solution containing the medium components other than yeast extract and sucrose was sterilized using an autoclave at 121° C. for 120 minutes. Finally, these three solutions were blended and filled to the mark with sterilized water to yield 500 mL of a medium.

Table 5 indicates sterile conditions for the respective media.

TABLE 4
Formulation Amount of Medium Components

| | Formulation |
|---|---|
| Medium components | ratio (w/v %) |
| Sucrose | 9.000 |
| Yeast extract (Product Name: YP-21CM, manufactured by | 5.000 |
| Fuji Foods Corporation) | |
| Yeast extract (Product Name: SK yeast extract HUP-2, | 2.000 |
| manufactured by NIPPON PAPER CHEMICALS) | |
| Decaglycerin monooleate (Product Name: Sunsoft Q-17S, | 0.300 |
| manufactured by Taiyo Kagaku Co., Ltd.) | |
| Sodium acetate trihydrate (which complies with food | 0.500 |
| additive standards) | |
| Dipotassium hydrogenphosphate (which complies with | 0.445 |
| food additive standards) | |
| Magnesium sulfate heptahydrate (which complies with food | 0.200 |
| additive standards) | |
| Purified water | 82.555 |
| Total | 100.000 |

TABLE 5
Sterile Conditions for Each Medium

| | | | Components other |
|---|---|---|---|
| | | | than yeast extract and |
| Medium | Yeast extract | Sucrose | sucrose |
| Example 4 | 121° C., 20 min | 121° C., 120 min | |
| Example 5 | 121° C., 20 min | 121° C., 120 min | 121° C., 120 min |

### (3) Culture of Lactic Acid Bacteria (Examples 6 and 7)

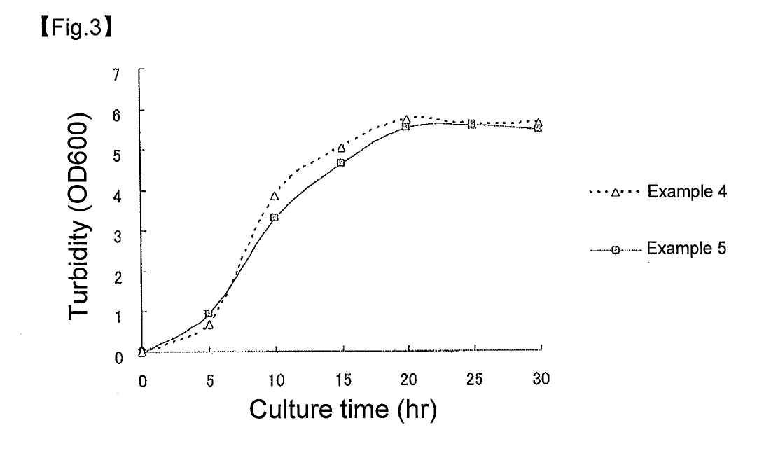

Lactic acid bacteria were cultured using the above respective media as obtained in Examples 4 and 5, in a procedure similar to that of Example 2. With regard to the turbidity, samples of a cultured medium were dispensed into a 96-well flat-bottom plate (manufactured by Nunc), and OD600 was determined using a multi-plate reader (manufactured by Dainippon Pharma Co., Ltd.) (FIG. 3).

Table 6 shows the results.

TABLE 6
Cell Yield of Powder of Microbial Cells Cultured by
Using Each Medium

| Cultured lactic acid | | |
|---|---|---|
| bacteria | Medium | Cell yield (g/L) |
| Example 6 | Medium of Example 4 | 7.8 |
| Example 7 | Medium of Example 5 | 7.8 |

FIG. 3 and Table 6 demonstrated that either the lactic acid bacteria (Example 6) which had been cultured using the medium (Example 4) prepared by independently sterilizing yeast extract alone and the other components or the lactic acid bacteria (Example 7) which had been cultured using the medium (Example 5) prepared by independently sterilizing yeast extract alone, sucrose alone, and the other components exhibited a better growth rate and cell yield.

### Examples 8 and 9

### Comparison of Sterilization Procedures in Terms of Sterilization of Nitrogen Source (Yeast Extract) Alone

### (1) Medium Preparation (Example 8, Comparative Example 3)

According to a formulation as designated in Table 7, the respective components were dissolved in purified water to prepare 500 mL of a medium. This medium was subjected to instantaneous sterilization by using a laboratory-scale UHT system (Model: 25HVH, manufactured by SEIKA CORPORATION) at 137° C. for 30 seconds.

### Comparative Example 3

Among the medium components as designated in Table 7, 25 g of yeast extract (YP-21 CM) and 10 g of yeast extract (HUP-2) were only dissolved in 115 mL of purified water to yield a yeast extract solution. In addition, medium components other than yeast extract were dissolved in purified water to 300 mL to yield a mixed solution containing the medium components other than yeast extract. Then, the above yeast extract solution was subjected to instantaneous sterilization by using a tube-type continuous sterilizer (a laboratory-scale UHT system, 25HVH, manufactured by SEIKA CORPORATION) at 137° C. for 30 seconds. In addition, the mixed solution containing the medium components other than yeast extract was sterilized using an autoclave at 121° C. for 20 minutes.

For any of the above sterilization procedures, the degree of sterilization was set to about F0=20.

Finally, the respective sterilized solutions as obtained above were blended and filled to the mark with sterilized water to yield 500 mL of a medium (Example 8).

Table 8 shows sterile conditions and sterilization procedures for the respective media.

TABLE 7
Formulation Amount of Medium Components

| | Formulation |
|---|---|
| Medium components | ratio (w/v %) |
| Sucrose | 9.000 |
| Yeast extract (Product Name: YP-21CM, manufactured by | 5.000 |
| Fuji Foods Corporation) | |
| Yeast extract (Product Name: SK yeast extract HUP-2, | 2.000 |
| manufactured by NIPPON PAPER CHEMICALS) | |
| Decaglycerin monooleate (Product Name: Sunsoft Q-17S, | 0.300 |
| manufactured by Taiyo Kagaku Co., Ltd.) | |
| Sodium acetate trihydrate (which complies with food | 0.500 |
| additive standards) | |
| Dipotassium hydrogenphosphate (which complies with | 0.445 |
| food additive standards) | |
| Magnesium sulfate heptahydrate (which complies with food | 0.200 |
| additive standards) | |
| Purified water | 82.555 |
| Total | 100.000 |

TABLE 8
Medium Sterile Conditions and Sterilization Procedure

| Medium | Sterile conditions and sterilization procedure |
|---|---|
| Comparative | Blend all the components, and perform UHT |
| Example 3 | sterilization at 137° C. for 30 sec. |
| Example 8 | Perform UHT sterilization of yeast extract alone at |
| | 137° C. for 30 sec. |
| | Perform autoclave sterilization of components other |
| | than yeast extract at 121° C. for 20 min |

### (2) Culture of Lactic Acid Bacteria

Lactic acid bacteria were cultured in the same conditions as in Example 2 except using the above media obtained in Comparative Example 3 and Example 8.

As an index for the cell number, the turbidity (OD600) at the end of the culture was determined. In addition, with regard to the resulting respective powder, the cell yield (g/L) per medium was estimated. Table 9 shows the results.

TABLE 9
Cell Yield of Powder of Microbial Cells Cultured by
Using Each Medium

| | | | Cell |
|---|---|---|---|
| Cultured lactic | | Turbidity at the end | yield |
| acid bacteria | Medium | of culture (OD600) | (g/L) |
| Comparative | Medium of Comparative | 47.5 | 8.1 |
| Example 4 | Example 3 | | |
| Example 9 | Medium of Example 8 | 48.9 | 8.9 |

Table 9 demonstrated that the lactic acid bacteria (Example 9) which had been cultured using the medium of Example 8 had a higher cell number and a higher cell yield than those (Comparative Example 4) which had been cultured using the medium of Comparative Example 3. In view of the above, effects of the present invention have been recognized even in the case of the UHT sterilization which seems to cause less denaturation of a medium.

### (3) Determination of Activities of Inducing IL-12 (Example 17 and Comparative Example 8)

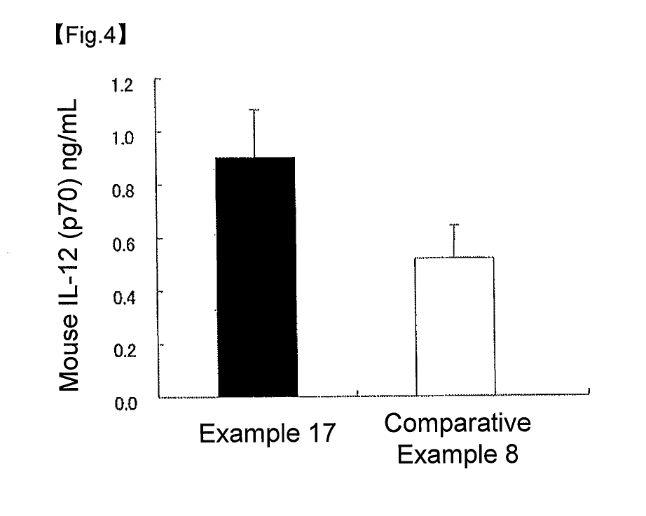

Activities of inducing IL-12 were determined (Example 17 and Comparative Example 8) using the same conditions as in Example 3 except using the above lactic acid bacteria as obtained in Example 9 or Comparative Example 4. FIG. 4 shows the results.

FIG. 4 demonstrated that the lactic acid bacteria (Example 9) which had been cultured using the medium (Example 8) prepared by separately sterilizing a yeast extract component and the other components and thereafter by blending them had a remarkably higher activity of inducing IL-12 (Example 17) than an activity of inducing IL-12 (Comparative Example 8) of the lactic acid bacteria(Comparative Example 4) which had been cultured using the medium (Comparative Example 3) prepared by blending all the medium components and thereafter by sterilizing them.

### Examples 10 to 12

### Large Scale Sterilization of Sugar (Sucrose) Alone

### (1) Medium Preparation (Example 10, Comparative Example 5)

According to a formulation as described in Table 1, the respective components were dissolved using a jar fermenter (manufactured by Hokko Kakouki Co., Ltd.) in 4.2 t of sterilized water (after sterilization with a 0.5-1 μm filter, the water was sterilized at 121° C. for 20 minutes). Then, batch sterilization using a steam injection process was carried out. The degree of sterilization was intended to be set to F0=20. The degree of sterilization was regulated using a thermo processor (Model: CMC821, manufactured by Ellab Corp.) (Comparative Example 5).

In addition, among the components listed in Table 1, 294 kg of sucrose was solely dissolved at 50% by weight with sterilized water to yield a sucrose solution. In addition, components listed in Table 1 except for sucrose were dissolved in 3152 L of sterilized water to yield a mixed solution containing the medium components other than sucrose. Next, the above sucrose solution and the mixed solution containing the medium components other than sucrose were each independently subjected to batch sterilization using a steam injection process. The degree of sterilization was intended to be set to F0=20. The degree of sterilization was regulated using a thermo processor. Then, these solutions after the sterilization were blended to yield 4.2 t of a medium. After that, the pH was adjusted to 6.8 by using about 50% by weight of aqueous sodium hydroxide (which complies with food additive standards) (Example 10).

With regard to the respective media, light absorbance at 600 nm (OD600) was determined to check a rough indication for medium color. Table 10 shows the results.

TABLE 10
Medium Sterile Conditions and Medium Color Tone after Sterilization

| | Sterile | | Color tone |
|---|---|---|---|
| Medium | conditions | Sterilization procedure | (OD600) |
| Comparative | Batch | Blend all the | 0.70 |
| Example 5 | sterilization | components, and sterilize | |
| Example 10 | Batch | Separately sterilize a | 0.44 |
| | sterilization | sucrose component and | |
| | | the other components | |

Table 10 demonstrated that the medium of Example 10 had a lower absorbance than the medium of Comparative Example 5. This suggests that the browning is suppressed.

### (2) Culture of Lactic Acid Bacteria (Example 11 and Comparative Example 6)

First, according to a formulation as described in Table 11, a medium was prepared by dissolving the respective components into sterilized water. By using this medium, lactic acid bacteria, a Lactobacillus acidophilus L-92 strain (FERM BP-4981), were cultured.

TABLE 11
Formulation Amount of Medium Components

| | Formulation |
|---|---|
| Medium components | ratio (w/v %) |
| Sucrose | 2.250 |
| Fish extract (Product Name: Bacterio-N-KS(B), | 1.000 |
| manufactured by Maruha Nichiro Seafoods, Inc.) | |
| Casein peptone (Product Name: Casein Peptone Plus, | 1.000 |
| manufactured by Organotechnie, Inc.) | |
| Yeast extract (Product Name: Yeast Peptone Standard | 0.500 |
| Type F, manufactured by Organotechnie, Inc.) | |
| Decaglycerin monooleate (Product Name: Sunsoft Q-17S, | 0.100 |
| manufactured by Taiyo Kagaku Co., Ltd.) | |
| Sodium acetate trihydrate (which complies with food | 0.500 |
| additive standards) | |
| Dipotassium hydrogenphosphate (which complies with | 0.445 |
| food additive standards) | |
| Magnesium sulfate heptahydrate (which complies with food | 0.100 |
| additive standards) | |
| Sterilized water | 94.105 |
| Total | 100.000 |

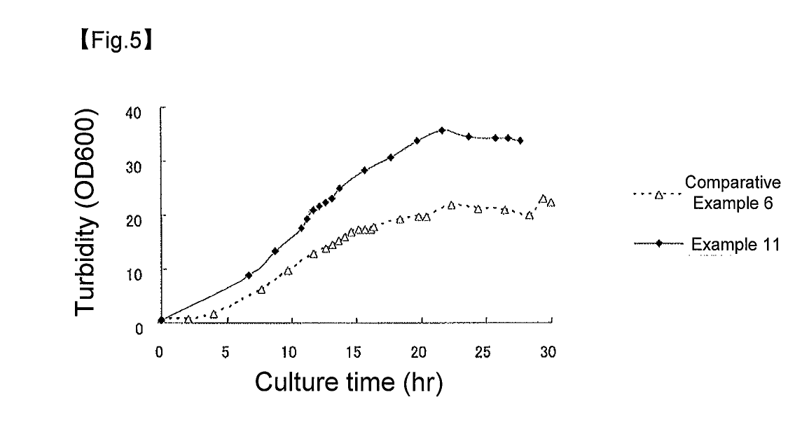

Next, 1 to 5% of the resulting lactic acid bacteria were aseptically inoculated in 4.2 t of the above respective media as obtained in Comparative Example 5 and Example 10. Until the pH reached 4.5 or lower (i.e., it took about 30 hours), the media were each cultured at a temperature of 37° C. with stirring. A cultured medium was sampled over time, and the turbidity (OD600) was determined to check a rough indication for the cell number (FIG. 5). After the culture, a cultured medium was removed by using a centrifuge (Model: SC-1, manufactured by GEA Westfalia Separator Japan K.K.), and a precipitate (microbial cells) was collected. The resulting microbial cells were washed with 4.2 t of sterilized water, sterilized using a plate-type continuous sterilizer (Model: SR2-P1, manufactured by APV Corp.) at 85° C. for several seconds, and powderized using a spray dryer (Model: SD-100R, manufactured by GEA Process Engineering K.K.). Finally, their color tone (the L value) was determined (Table 12).

TABLE 12
Turbidity at the End of Culture and Color Tone of
Microbial Cell Powder

| Cultured | | Turbidity at the | Color |
|---|---|---|---|
| lactic acid | | end of culture | tone (L |
| bacteria | Medium | (OD600) | Value) |
| Comparative | Medium of Comparative | 22.4 | 62.1 |
| Example 6 | Example 5 | | |
| Example 11 | Medium of Example 10 | 33.9 | 79.4 |

FIG. 5 and Table 12 demonstrated that even in the case of a large scale of 4.2 t, the lactic acid bacteria (Example 11) which had been cultured using the medium of Example 10 had a higher growth rate of the lactic acid bacteria as well as a higher cell content (OD600) than those (Comparative Example 6) which had been cultured using the medium of Comparative Example 5. In addition, regarding the color tone indicated by the L value, the lactic acid bacteria of Example 11 had a larger L value, which demonstrated that the lactic acid bacteria of Example 11 had less browning than the lactic acid bacteria of Comparative Example 6. This also indicated that lactic acid bacteria powder having a better color tone was obtained.

### (3) Determination of Activities of Inducing IL-12 (Example 12 and Comparative Example 7)

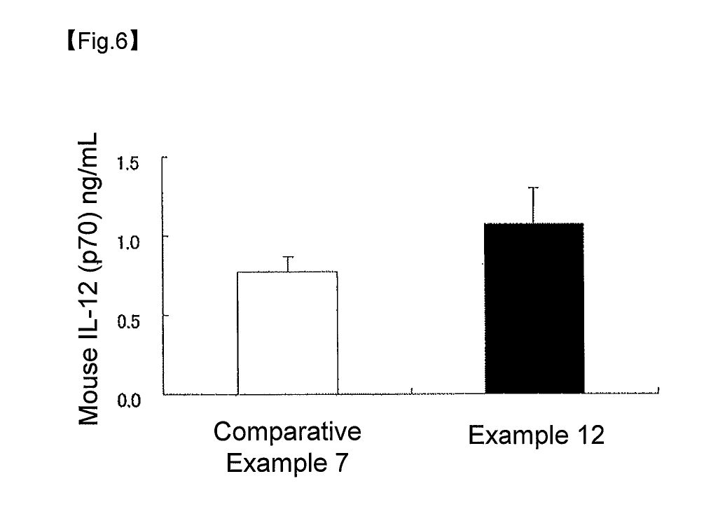

In the meantime, after the culture, 50 mL of the cultured medium was separately centrifuged (at 3000×g, for 10 minutes, and at room temperature) at a laboratory level to remove a supernatant. Then, a precipitate (microbial cells) was washed with 50 mL of purified water. After the washing, the microbial cells which were powderized by lyophilization were used as a separate stock. Then, an activity of inducing IL-12 was determined using this stock under conditions similar to those of Example 3 (FIG. 6).

FIG. 6 demonstrated that the lactic acid bacteria (Example 11) which had been cultured using the medium (Example 10) prepared by separately sterilizing a sucrose component and the other components and thereafter by blending them had a higher activity of inducing IL-12 (Example 12) than an activity of inducing IL-12 (Comparative Example 7) of the lactic acid bacteria (Comparative Example 6) which had been cultured using the medium (Comparative Example 5) prepared by blending all the medium components and thereafter by sterilizing them.

### Examples 13 to 16

### Sterilization of Nitrogen Source (Yeast Extract) Alone

### (1) Medium Preparation, Part I (Example 13)

Among the medium components as designated in Table 13, 45 g of sucrose was solely dissolved in 55 mL of purified water to yield a sucrose solution. In addition, medium components other than sucrose were dissolved in purified water to 400 mL to yield a mixed solution containing the medium components other than sucrose. Then, the above sucrose solution was sterilized using an autoclave at 121° C. for 20 minutes. Also, after the mixed solution containing the medium components other than sucrose had been sterilized using an autoclave at 121° C. for 20 minutes, these solutions were mixed and filled to the mark with sterilized water to yield 500 mL of a medium. Table 14 shows the sterile conditions.

### (2) Medium Preparation, Part II (Example 14)

Among the medium components listed in Table 13, 25 g of yeast extract (YP-21CM) and 10 g of yeast extract (Yeast peptone MAX) were only dissolved in 115 mL of purified water to yield a yeast extract solution. Further, medium components other than yeast extract were dissolved in purified water to 350 mL to yield a mixed solution containing the medium components other than yeast extract. Then, the above yeast extract solution was sterilized using an autoclave at 121° C. for 20 minutes. In addition, the above mixed solution containing the medium components other than yeast extract was sterilized using an autoclave at 121° C. for 20 minutes. Finally, these solutions were blended and filled to the mark with sterilized water to yield 500 mL of a medium.

Table 14 shows the sterile conditions for the respective media.

TABLE 13
Formulation Amount of Medium Components

| | Formulation |
|---|---|
| Medium components | ratio (w/v %) |
| Sucrose | 9.000 |
| Yeast extract (Product Name: YP-21CM, manufactured by | 5.000 |
| Fuji Foods Corporation) | |
| Yeast extract (Product Name: Yeast peptone MAX., | 2.000 |
| manufactured by Ohly Yeast) | |
| Decaglycerin monooleate (Product Name: Sunsoft Q-17S, | 0.300 |
| manufactured by Taiyo Kagaku Co., Ltd.) | |
| Sodium acetate trihydrate (which complies with food | 0.500 |
| additive standards) | |
| Dipotassium hydrogenphosphate (which complies with | 0.445 |
| food additive standards) | |
| Magnesium sulfate heptahydrate (which complies with food | 0.200 |
| additive standards) | |
| Purified water | 82.555 |
| Total | 100.000 |

TABLE 14
Sterile Conditions for Each Medium

| | |
|---|---|
| Example 13 | Sucrose Alone: 121° C., 20 min |
| | Mixture containing medium components other than |
| | sucrose: 121° C., 20 min |
| Example 14 | Yeast extract alone: 121° C., 20 min |
| | Mixture containing medium components other than yeast |
| | extract: 121° C., 20 min |

### (3) Culture of Lactic Acid Bacteria (Examples 15 and 16)

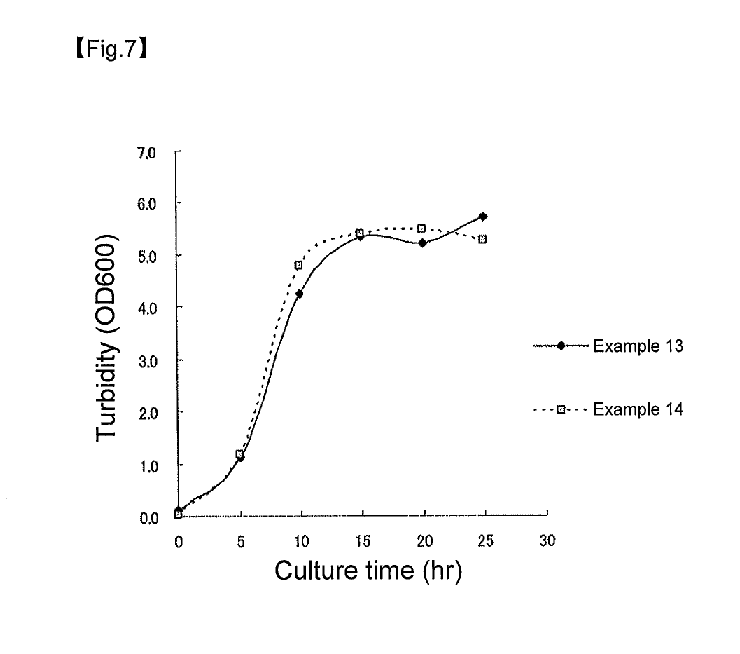

Lactic acid bacteria were cultured using the above respective media as obtained in Examples 13 and 14, in a procedure similar to that of Example 2. With regard to the turbidity, samples of a cultured medium were dispensed into a 96-well flat-bottom plate (manufactured by Nunc), and OD600 was determined using a multi-plate reader (manufactured by Dainippon Pharma Co., Ltd.) (FIG. 7).

### (4) Determination of Activities of Inducing IL-12

Activities of inducing IL-12 were determined using the same conditions as in Example 3 except using the above lactic acid bacteria as obtained in Example 13 or 14. Table 15 shows the results.

TABLE 15
Cell Yield of Powder of Microbial Cells Cultured by
Using Each Medium

| Cultured lactic acid | | | Activity of inducing |
|---|---|---|---|
| bacteria | Medium | Cell yield (g/L) | IL-12 (ng/mL) |
| Example 15 | Medium of | 7.73 | 1.05 |
| | Example 13 | | |
| Example 16 | Medium of | 7.75 | 1.19 |
| | Example 14 | | |

Table 14, FIG. 7, and Table 15 demonstrated that either the lactic acid bacteria (Example 15) which had been cultured using the medium (Example 13) prepared by independently sterilizing sucrose alone and the other components or the lactic acid bacteria (Example 16) which had been cultured using the medium (Example 14) prepared by independently sterilizing yeast extract alone and the other components, exhibited a better growth rate and cell yield. This resulted in microbes having a better activity of inducing IL-12.

These Examples specifically revealed the following in particular.

A browning phenomenon called a Maillard reaction has been known as an interaction between medium nutritional components. This browning phenomenon was not thought to cause a problem during sterilization of a mixture containing a nonreducing sugar such as sucrose and a nitrogen source. However, as disclosed in an invention of the present application, a higher growth rate of microbes as well as a higher cell yield can be achieved by separate sterilization of a nonreducing sugar and a nitrogen source. This method has also allowed for a good color tone and has further definitely produced microbes having an elevated immunoregulatory function.

1. A method for producing a medium for culturing microbes, comprising the steps of:
(1) sterilizing a solution comprising a sugar source material;
(2) sterilizing a solution comprising a nitrogen source material; and
(3) blending the two solutions as obtained in steps (1) and (2).

2. A method for producing a medium for culturing microbes, comprising the steps of:
(1) sterilizing a solution devoid of a nitrogen source, the solution comprising a sugar;
(2) sterilizing a solution devoid of a sugar, the solution comprising a nitrogen source; and
(3) blending the two solutions as obtained in steps (1) and (2).

3. A method for producing a medium for culturing microbes, comprising the steps of:
(1) sterilizing a solution solely comprising a sugar;
(2) sterilizing a solution solely comprising a nitrogen source;
(3) sterilizing a solution devoid of a sugar or a nitrogen source, the solution comprising at least one selected from the group consisting of inorganic salts, vitamins, fatty acids, buffers, and antifoaming agents; and
(4) blending the three solutions as obtained in steps (1), (2), and (3).

4. The method for production according to claim 1, wherein the sugar source material or sugar is a nonreducing sugar.

5. The method for production according to claim 4, wherein the nonreducing sugar comprises at least one selected from the group consisting of sucrose, trehalose, kestose, melezitose, gentianose, neobifurcose, fungitetraose, and bifurcose.

6. The method for production according to claim 4, wherein the nonreducing sugar is sucrose.

7. The method for production according to claim 1, wherein the nitrogen source material or nitrogen source comprises at least one selected from the group consisting of amino acids, peptides, proteins, urea, casein hydrolysates, corn steep liquor, soy bean, soy bean hydrolysates, peanut meal, cotton seed meal, fish meal, fish extract, beef extract, and yeast extract.

8. The method for production according to claim 1, wherein the step of sterilizing a solution comprising a sugar source material or the step of sterilizing a solution comprising a sugar is carried out by batch sterilization and/or continuous sterilization.

9. The method for production according to claim 1, wherein the step of sterilizing a solution comprising a nitrogen source material or the step of sterilizing a solution comprising a nitrogen source is carried out by batch sterilization and/or continuous sterilization.

10. The method for production according to claim 3, wherein batch sterilization and/or continuous sterilization is carried out in the step of sterilizing a solution devoid of a sugar or a nitrogen source, the solution comprising at least one selected from the group consisting of inorganic salts, vitamins, fatty acids, buffers, and antifoaming agents.

11. A medium which is produced by the method according to claim 1.

12. A method for culturing microbes, comprising the step of using the medium according to claim 11.

13. The method for culture according to claim 12, wherein the microbes are lactic acid bacteria.

14. Microbes which are cultured by the method for culture according to claim 12.

15. Lactic acid bacteria which are cultured by the method for culture according to claim 13.

16. A method for producing a medium for producing an immunomodulator, comprising the steps of:
(1) sterilizing a solution comprising a sugar source material;
(2) sterilizing a solution comprising a nitrogen source material; and
(3) blending the two solutions as obtained in steps (1) and (2).

17. A method for producing a medium for producing an immunomodulator, comprising the steps of:
(1) sterilizing a solution devoid of a nitrogen source, the solution comprising a sugar;
(2) sterilizing a solution devoid of a sugar, the solution comprising a nitrogen source; and
(3) blending the two solutions as obtained in steps (1) and (2).

18. A method for producing a medium for producing an immunomodulator, comprising the steps of:
(1) sterilizing a solution solely comprising a sugar;
(2) sterilizing a solution solely comprising a nitrogen source;
(3) sterilizing a solution devoid of a sugar or a nitrogen source, the solution comprising at least one selected from the group consisting of inorganic salts, vitamins, fatty acids, buffers, and antifoaming agents; and
(4) blending the three solutions as obtained in steps (1), (2), and (3).

19. The method for production according to claim 16, wherein the sugar source material or sugar is a nonreducing sugar.

20. The method for production according to claim 19, wherein the nonreducing sugar comprises at least one selected from the group consisting of sucrose, trehalose, kestose, melezitose, gentianose, neobifurcose, fungitetraose, and bifurcose.

21. The method for production according to claim 19, wherein the nonreducing sugar is sucrose.

22. The method for production according to claim 16, wherein the nitrogen source material or nitrogen source comprises at least one selected from the group consisting of amino acids, peptides, proteins, urea, casein hydrolysates, corn steep liquor, soy bean, soy bean hydrolysates, peanut meal, cotton seed meal, fish meal, fish extract, beef extract, and yeast extract.

23. The method for production according to claim 16, wherein the step of sterilizing a solution comprising a sugar source material or the step of sterilizing a solution comprising a sugar is carried out by batch sterilization and/or continuous sterilization.

24. The method for production according to claim 16, wherein the step of sterilizing a solution comprising a nitrogen source material or the step of sterilizing a solution comprising a nitrogen source is carried out by batch sterilization and/or continuous sterilization.

25. The method for production according to claim 18, wherein batch sterilization and/or continuous sterilization is carried out in the step of sterilizing a solution devoid of a sugar or a nitrogen source, the solution comprising at least one selected from the group consisting of inorganic salts, vitamins, fatty acids, buffers, and antifoaming agents.

26. A medium which is produced by the method for production according to claim 16.

27. A method for producing an immunomodulator, comprising the step of using the medium according to claim 26.

28. The method for production according to claim 27, wherein the immunomodulator is an antiallergic agent.

29. The method for production according to claim 27, wherein the immunomodulator is an activator for inducing IL-12.

30. An immunomodulator which is produced by the method for production according to claim 27.

31. An antiallergic agent which is produced by the method for production according to claim 28.

32. An activator for inducing IL-12, the activator being produced by the method for production according to claim 29.
